(19) United States
(12) Patent Application Publication
(10) Pub. No.: US 2010/0217139 A1
(43) Pub. Date: Aug. 26, 2010

(54) HEART RATE MEASUREMENT

(75) Inventors: Robert Pinter, Aachen (DE); Elke Naujokat, Aachen (DE); Claudia Hannalore Igney, Aachen (DE); Guido J. Muesch, Erkelenz (DE)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS N.V., Eindhoven (NL)

(21) Appl. No.: 12/095,513

(22) PCT Filed: Nov. 30, 2006
(86) PCT No.: PCT/IB2006/054524

§ 371 Date: May 30, 2008

(30) Foreign Application Priority Data
Nov. 30, 2005 (EP) 05111496.5

(51) Int. Cl. A61B 5/02

(52) U.S. Cl. 600/508

(57) ABSTRACT

The invention relates to a monitoring device (105) for remotely monitoring a heartbeat of a subject, the monitoring device comprising a remote sensor (102) for receiving a monitored signal (103) indicative of a movement of the subject's thoracic wall (101) induced by the subject's heartbeat. In an embodiment of the invention, the monitoring device (105) further comprises a triggering device (401) arranged to access the monitored signal (403) to generate a triggering signal (402) representative of a phase of the heartbeat. The invention further relates to an imaging or spectroscopy system (901), for example a magnetic resonance or computed tomography system or a cardiac 3D X-ray angiography system, arranged to acquire data from a subject, the system comprising such a monitoring device (105), wherein the system is further arranged to utilize the triggering signal (402) to synchronize the acquisition of the data to the phase of the subject's heartbeat.

The invention relates to a monitoring device for remotely monitoring a heartbeat of a subject, the monitoring device being arranged to receive a monitored signal indicative of a movement of the subject.

The invention further relates to an imaging or spectroscopy system, for example a magnetic resonance (MR) or computed tomography (CT) system or a cardiac three-dimensional (3D) X-ray angiography system, arranged to acquire data from a subject, the system comprising such a monitoring device, wherein the system is further arranged to utilize a triggering signal to synchronize the acquisition of the data to a phase of the subject's heartbeat.

The invention further relates to a method of remote monitoring of a heartbeat of a subject, the monitoring being based on a monitored signal indicative of a movement of the subject, the monitored signal being sensed remotely.

The invention further relates to a computer program product comprising instructions

to access a monitored signal that is remotely received by a monitoring device, the monitored signal being representative of a subject's heartbeat,

to actuate a triggering device to generate a triggering signal based on the monitored signal, the triggering signal being representative of a phase of the subject's heartbeat, and

to synchronize data acquisition on an imaging or spectroscopy system to the phase of the subject's heartbeat, the synchronization being effected by the triggering signal,

when the computer program product is run on a computer.

An embodiment of a device implementing such a method is discussed in U.S. Pat. No. 5,573,012, which teaches a method and an apparatus to monitor the movement of internal body parts, such as the heart. The embodiment involves the emission and detection of very short, voltage pulses by employing pulse-echo radar in repetitive mode, and clocking the two-way time of flight of the electromagnetic (EM) pulse. A large number of reflected pulses are averaged to produce a voltage that is modulated by reflections from the heart wall.

A problem with the prior art is that the method implemented by the device is rather cumbersome. It is thus an object of the invention to provide a device that implements a less cumbersome technique to monitor a subject's heartbeat.

This object is achieved by a monitoring device according to the first paragraph, wherein the monitored signal is received from an external surface of the subject's thoracic wall. Unlike the prior art, where the device needs to directly monitor the heart wall or tissue in order to detect the heartbeat, the current invention remotely senses the subject's heartbeat by detecting the effect of the heartbeat on the thoracic wall, using various remote sensing techniques. Examples of such non-contact techniques include capturing stereoscopic pictures, high-resolution video, etc.

This and other aspects of the invention will be elaborated further on the basis of the following embodiments, which are defined in the dependent claims.

An embodiment of the monitoring device according to the invention further comprises a processor for processing the monitored signal to generate an output signal indicative of the heartbeat. Though the monitored signal comprises information about the heartbeat, it may sometimes be necessary to process the monitored signal to convert it to an output signal so that it can be input to other devices. For example, the processor could convert the monitored signal into a current or voltage signal that is displayed as a waveform on a screen or a monitor. The processor could also, for instance, average multiple heartbeats over a specified time period, and output a signal indicative of an average heart rate. Such an average heart rate could then be used to predict when the next heartbeat may occur.

In a further embodiment, the monitoring device according to the invention further comprises a transmitter for transmitting a measurement signal towards the subject's thoracic wall, wherein, when in operation, the measurement signal interacts with the thoracic wall to generate the monitored signal. The transmitter is located at a suitable distance from the patient, and is arranged to transmit radiation, for example EM radiation, ultrasound, etc., towards the patient. The transmitted radiation is reflected from the subject's thoracic wall, thereby generating the monitored signal. Alternatively, the transmitted radiation interacts with the chest wall to produce a different radiation that could form the monitored signal. For example, a material that fluoresces when exposed to light of a certain wavelength could be tightly draped over the patient, or even painted on the chest wall. When the fluorescent material is excited by incident light, the fluorescence may be detected to monitor heart motion.

In a further embodiment, the monitoring device according to the invention further comprises a triggering device arranged to access the monitored signal to generate a triggering signal representative of a phase of the heartbeat. The triggering device may generate the triggering signal corresponding to a particular phase of the heartbeat, for example a ventricular contraction phase or an atrial diastolic phase, etc. The triggering signal could be in the form of a current or voltage pulse, or an optical pulse etc., which can in turn be used to trigger the next step in the monitoring process.

In a further embodiment of the monitoring device according to the invention, the measurement signal is EM radiation, the monitored signal is reflected EM radiation, and the output signal is the Doppler shift in frequencies between the measurement signal and the monitored signal. For example, a microwave transceiver emits a continuous-wave microwave beam as the measurement signal towards the thorax and receives the reflection from the thoracic wall as the monitored signal. The reflection of a wave at moving surfaces causes a frequency shift in the reflected signal compared to the transmitted signal. The magnitude of the frequency shift is representative of the motion of the reflecting surface. Thus, by measuring the Doppler shift in frequencies between the transmitted and the reflected EM waves, the effect of the heartbeat on the thoracic wall can be isolated, thus permitting monitoring of the heartbeat in a non-contact manner.

In a further embodiment of the monitoring device according to the invention, the measurement signal is optical EM radiation, for example a beam of light, the monitored signal is reflected optical EM radiation that is processed to yield a time series of shearograms of the subject's thoracic wall and the output signal is obtained by comparing consecutive shearograms in the time series. Instead of an ordinary beam of light, a monochromatic light source like a laser may also be used to transmit the measurement signal. By means of an optical system, a series of images of the thorax is generated. Each image of the object, in this case the thorax, is further duplicated, for instance by optical means, and at the same time shifted and superimposed on the original image. This creates the impression of a shearing strain on the image, and the resulting image is called a shearogram.

Shearography is a relative measuring method, in which the resulting image represents the difference between two states of the recorded object shifted in time. Every shearogram is compared to, for example, its preceding shearogram, to produce a comparison image. If the optical path lengths of two pixels change to the same extent or not at all, no differential information can be derived. However, if the location of a pixel changes with respect to that of a neighbouring pixel, this difference in optical path length leads to quantitative information about a local change, which in turn leads to local specks or to stripe patterns in the comparison image. These local specks or stripe patterns are indicative of the effect of the heartbeat on the thoracic wall. The concept of shearography is further explained in “Digital Shearography: Theory and Application of Digital Speckle Pattern Shearing Interferometry” by Wolfgang Steinchen, Lianxiang Yang, published by SPIE-International Society for Optical Engine (February 2003).

In a further embodiment of the monitoring device according to the invention, the monitored signal is optical EM radiation that is processed to yield a time series of stereoscopic images, and the output signal is obtained by comparing consecutive stereoscopic images in the time series. A stereoscopic camera monitors the patient's thorax. Though a single channel camera could be used instead, stereoscopy has the advantage of an enhanced assessment of the size, the distance and consequently also the movement of the monitored object. The small movements of the patient's thorax, caused by the beating of the heart, are registered by the stereoscopic camera. Consecutive image captures taken during the measurement show a change in image characteristics due to the movement of the thoracic wall, which is in turn caused by the motion of the heart.

In a further embodiment of the monitoring device according to the invention, the measurement signal is ultrasound radiation, the monitored signal is reflected ultrasound radiation, and the output signal is the Doppler shift between the measurement signal and the monitored signal. An ultrasound transmitter emits an ultrasonic beam as the measurement signal towards the thorax and an ultrasound receiver receives the reflections from the thoracic wall as the monitored signal. The reflection of a wave at moving surfaces causes a frequency shift in the reflected signal compared to the transmitted signal. The magnitude of the frequency shift is representative of the motion of the reflecting surface. Thus, by measuring the Doppler shift in frequencies between the transmitted and the reflected ultrasonic waves, the effect of the heartbeat on the thoracic wall can be isolated, thus permitting monitoring of the heartbeat in a non-contact manner.

It is a further object of the invention to provide an imaging or spectroscopy system as in the opening paragraphs, wherein the heartbeat of the subject is detected in a less cumbersome manner.

This object is achieved by an imaging or spectroscopy system according to the first paragraphs, wherein the monitored signal, used to generate the triggering signal, is received from an external surface of the subject's thoracic wall. The imaging or spectroscopy data acquisition system is setup such that the data acquisition is synchronized to a phase of the subject's heartbeat. For example, in an MR imaging system, data acquisition is synchronized such that a particular region or a particular line of k-space, or even the full k-space, is acquired during a particular phase of the heartbeat. For instance, the heart moves the least during its diastolic phase, and therefore a triggering signal indicative of this phase is used to trigger the acquisition of the central region of k-space, such that motion artifacts in the acquired image are minimized. During the ventricular contraction phase, when the heart moves the most, the triggering signal may trigger the acquisition of the outer lines of k-space. Alternatively, it is also possible to trigger the acquisition, for example on a CT scanner, such that an entire dataset is acquired during each trigger. For instance, image acquisition may be initiated at the end of every ventricular contraction phase, and allowed to continue so that an entire image is collected after each initiation. Similarly, it is also advantageous to synchronize image acquisition to a phase of the heartbeat in the case of cardiac three-dimensional x-ray angiography.

It is a further object of the invention to provide a less cumbersome method of monitoring a subject's heartbeat.

This object is achieved by a method according to the first paragraphs, wherein the monitored signal is received from an external surface of the subject's thoracic wall. The invention remotely senses the subject's heartbeat by detecting the effect of the heartbeat on the thoracic wall, using various remote sensing or non-contact techniques. Examples of such non-contact techniques include capturing stereoscopic pictures, high-resolution video, etc.

This and other aspects of the invention will be elaborated further on the basis of the following embodiments, which are defined in the dependent claims.

In an implementation of the method according to the invention, the monitored signal is processed to generate an output signal indicative of the heartbeat. Though the monitored signal comprises information about the heartbeat, it may often be necessary to process the monitored signal to convert it to an output signal so that it can be input to other devices. For example, the monitored signal could be processed and converted into a current or voltage signal that is displayed as a waveform on a screen or a monitor. Other examples of processing the monitored signal include filtering, amplification, conversion to optical signals, etc.

In a further implementation, the method according to the invention further comprises a step of transmitting a measurement signal towards the subject's thoracic wall, wherein the monitored signal is generated from interactions of the measurement signal with the thoracic wall. The transmitted radiation may comprise EM radiation, laser light, ultrasound, etc., which may be reflected from the subject's thoracic wall to generate the monitored signal. Alternatively, the transmitted radiation may interact with the chest wall to produce a different radiation that could form the monitored signal. For example, a material that fluoresces when exposed to EM radiation of a certain wavelength could be tightly draped over, painted on, or otherwise represented on the subject's chest wall. When the fluorescent material is excited by incident EM radiation, the fluorescence is detected to monitor heart motion in a contact-less fashion.

In a further implementation, the method according to the invention further comprises a step of using the monitored signal to generate a triggering signal representative of a phase of the subject's heartbeat. The triggering signal may correspond to a particular phase of the heartbeat, for example a ventricular contraction phase or an atrial diastolic phase, etc. Different types of triggering signals may comprise audible signals from an annunciator, electrical signals such as a voltage or current pulse, etc.

It is a further object of the invention to provide a computer program to be loaded by a computer arrangement, the computer program comprising instructions for synchronizing data acquisition on an imaging or spectroscopy system to a phase of a subject's heartbeat, wherein the subject's heartbeat is detected in a less cumbersome manner.

This object is achieved by a computer program product according to the opening paragraphs, wherein the monitored signal is remotely received from the external surface of the subject's thoracic wall. A monitoring device, for example a stereoscopic camera or a high-resolution video camera, receives the monitored signal. The computer program provides the capability to access the monitored signal. The computer program could also provide instructions to process the monitored signal, thereby generating a processed signal that is indicative of the subject's heartbeat. The computer program could alternatively provide instructions to a processor arranged to process the monitored signal, the processor generating a processed signal as the output. The computer program further provides instructions to control a triggering device that accepts the monitored signal or the processed signal, as its input. The triggering device outputs a triggering signal that is representative of a phase of the subject's heartbeat. The computer program could also provide instructions to identify the phase of the subject's heartbeat. The computer program further provides instructions to synchronize data acquisition on an imaging or spectroscopy system, the synchronization being based on the triggering signal. The computer program product could be a computer program residing on a computer-readable medium, for example a CD-ROM or a DVD. Alternatively, the computer program product could be a downloadable program that is downloaded, or otherwise transferred to the computer, for example via the Internet.

This and other aspects of the invention will be elaborated further on the basis of the following embodiments, which are defined in the dependent claims.

In an embodiment of the computer program product according to the invention, the computer program further provides instructions to control a transmitter capable of transmitting a measurement signal. The measurement signal interacts with the subject's thoracic wall to generate the monitored signal that is sensed by the monitoring device. The computer program could instruct the transmitter to initiate transmission of the measurement signal. The computer program could further control the intensity or the duration of the measurement signal.

These and other aspects of the invention will be described in detail hereinafter, by way of example, on the basis of the following embodiments, with reference to the accompanying drawings, wherein

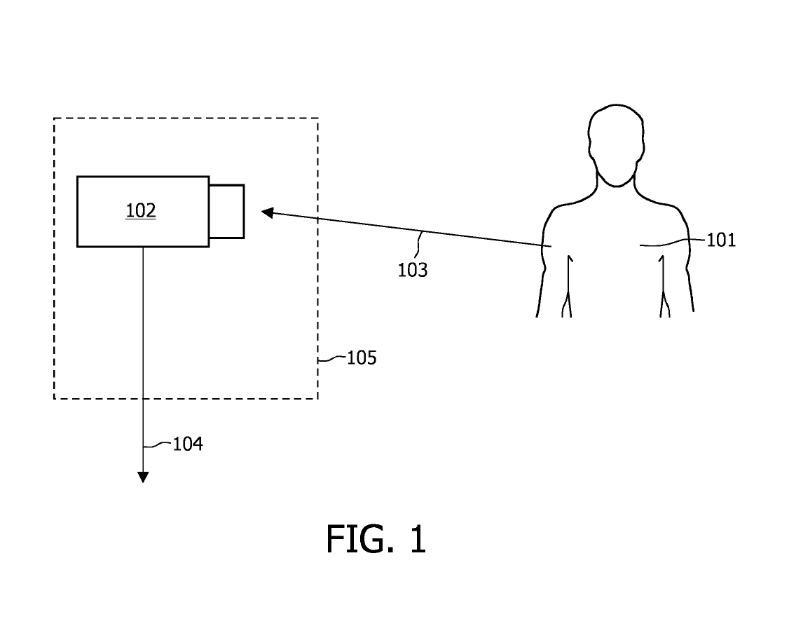

FIG. 1 schematically shows a device according to the invention,

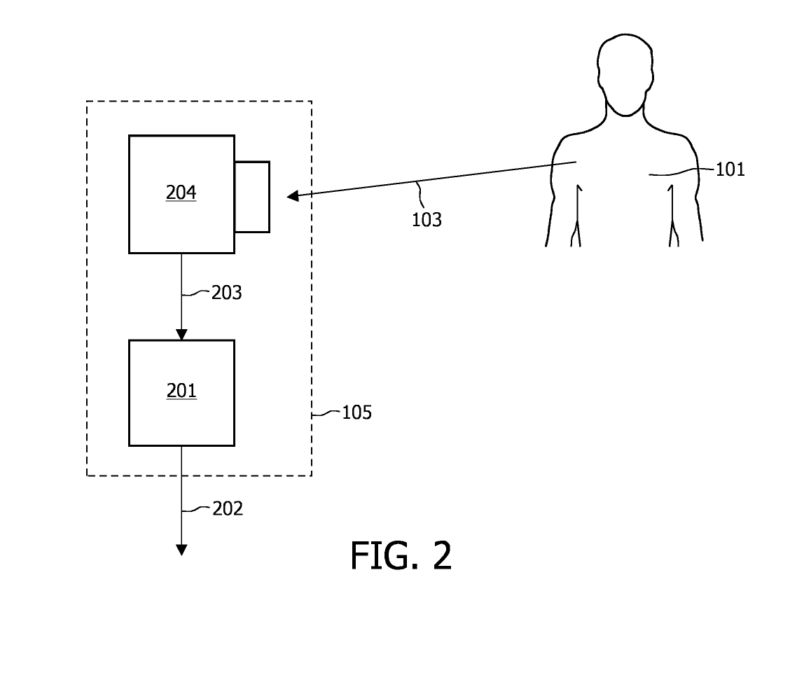

FIG. 2 schematically shows a device according to the invention, further comprising a processing unit,

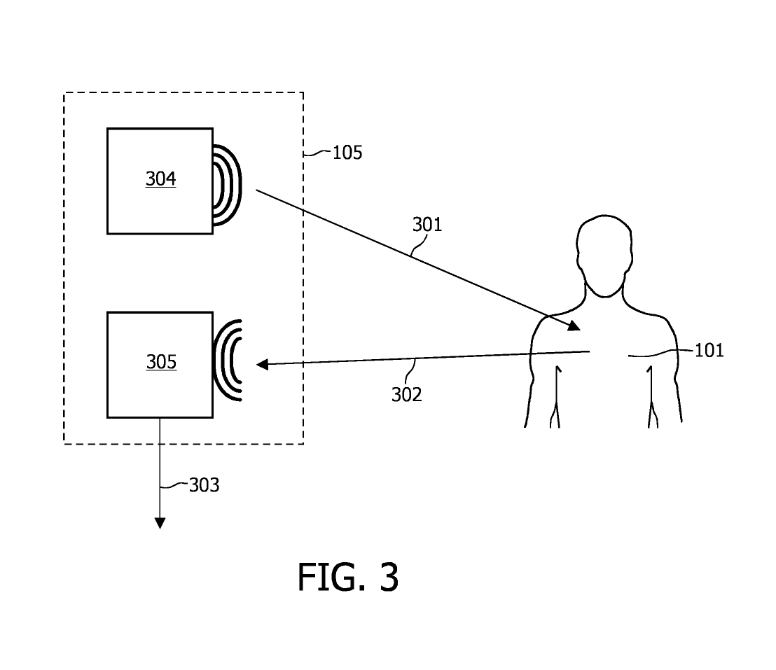

FIG. 3 schematically shows a device according to the invention, further comprising a transmitting unit,

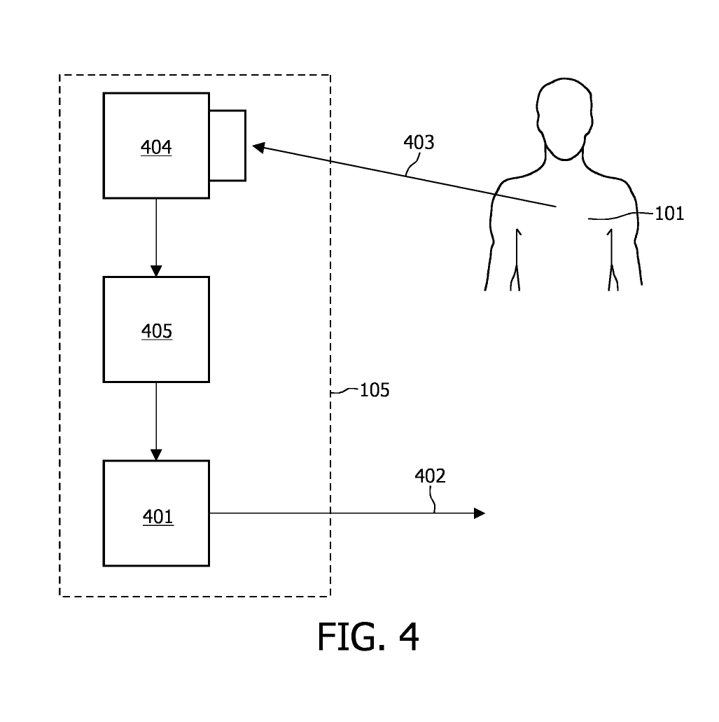

FIG. 4 schematically shows a device according to the invention, further comprising a triggering unit,

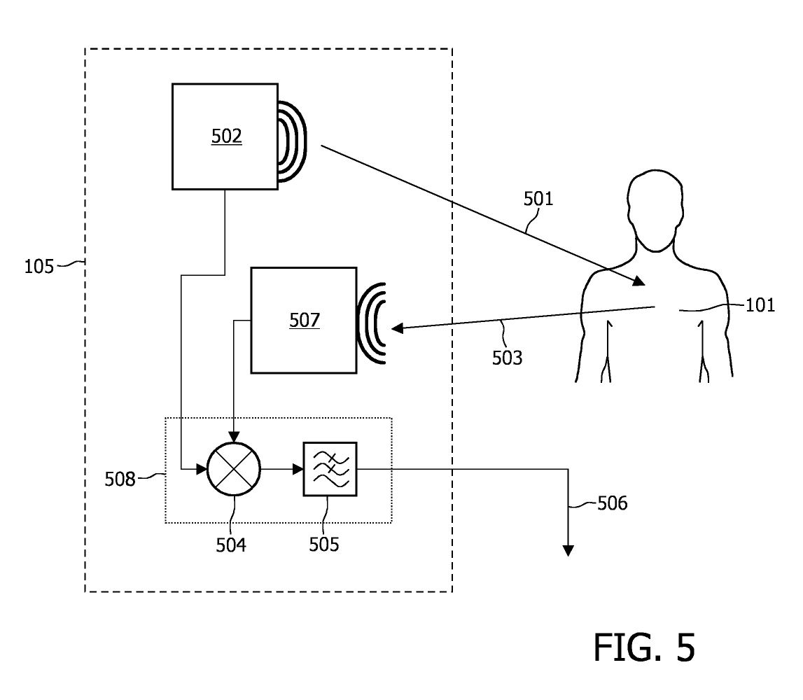

FIG. 5 schematically shows an embodiment of the device according to the invention, wherein the measurement signal is EM or ultrasonic radiation, the monitored signal is reflected EM or ultrasonic radiation, and the output signal is the Doppler shift in frequencies between the measurement signal and the monitored signal,

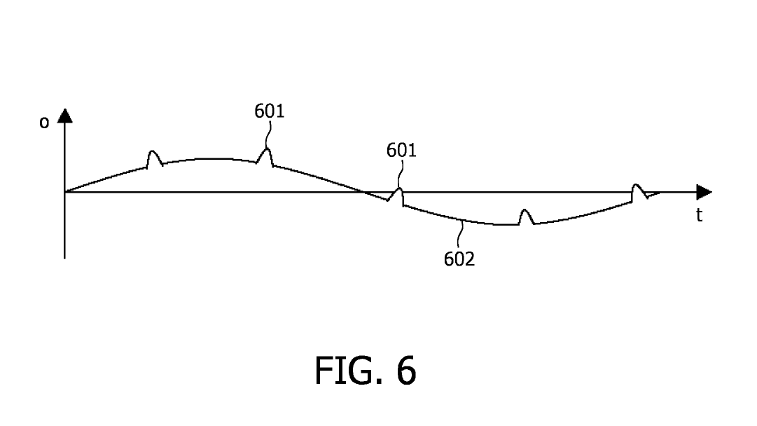

FIG. 6 diagrammatically shows the movement of the thoracic wall of a subject, wherein the effect of the patient's heartbeats is superimposed on respiratory motion, and wherein the axis labelled “o” represents the magnitude of displacement of the thoracic wall, and the axis labelled “t” represents time,

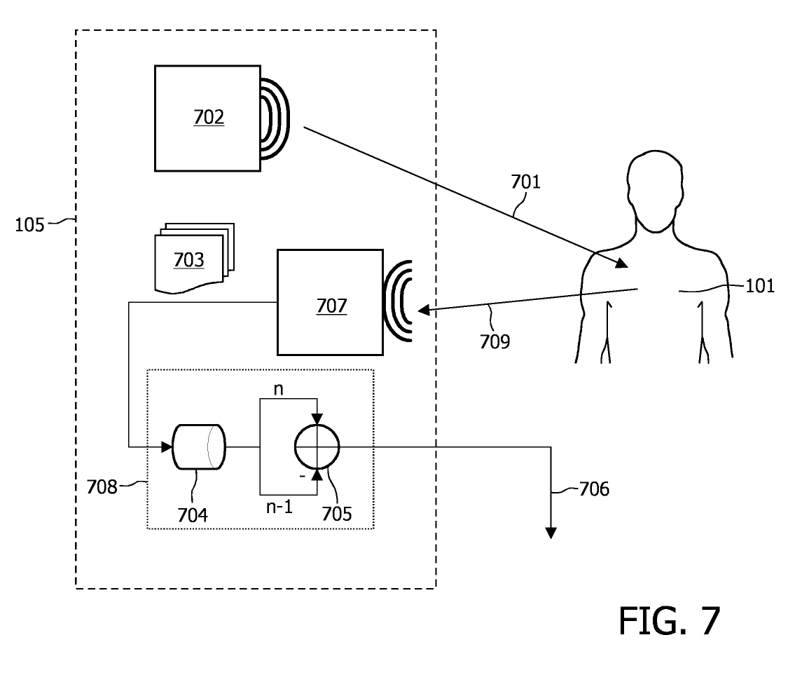

FIG. 7 schematically shows a further embodiment of the device according to the invention, wherein the measurement signal is optical EM radiation, the monitored signal is reflected optical EM radiation that is processed to yield a time series of shearograms of the subject's thoracic wall and, the output signal is obtained by comparing consecutive shearograms in the time series,

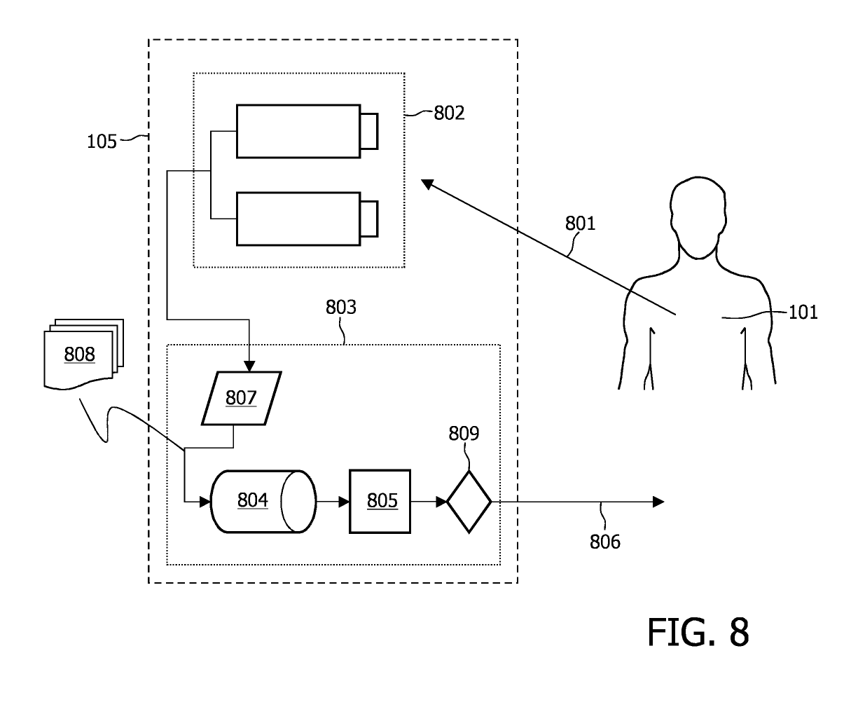

FIG. 8 schematically shows a further embodiment of the device according to the invention, wherein the monitored signal is optical EM radiation that is processed to yield a time series of stereoscopic images, and the output signal is obtained by comparing consecutive stereoscopic images in the time series,

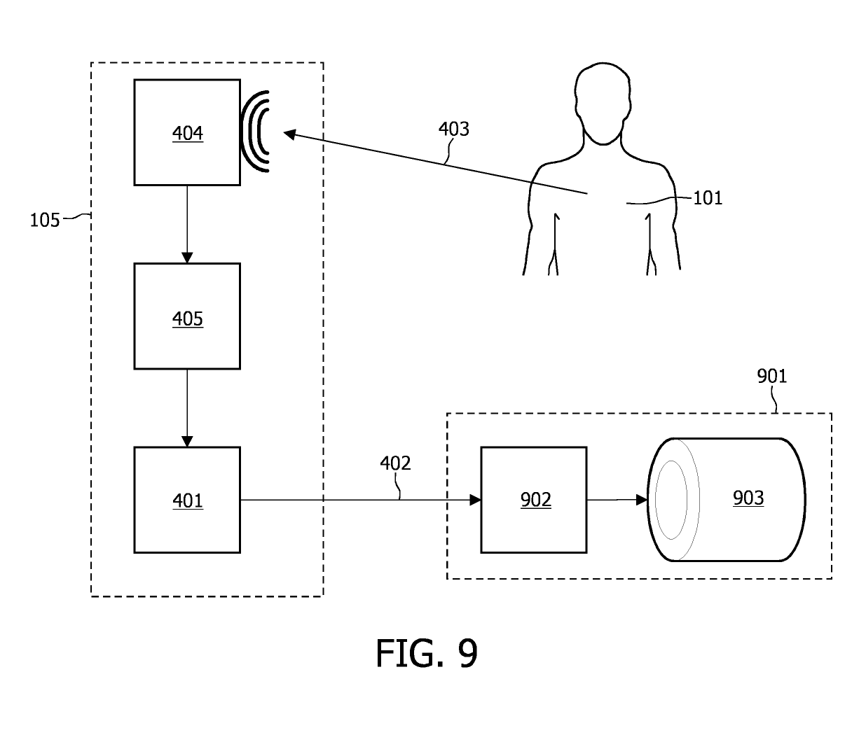

FIG. 9 schematically shows an embodiment of an imaging or spectroscopy system arranged to acquire data from a subject, the system comprising a monitoring device according to an embodiment of the invention, wherein the system is further arranged to utilize a triggering signal to synchronize the acquisition of the data to a phase of the subject's heartbeat,

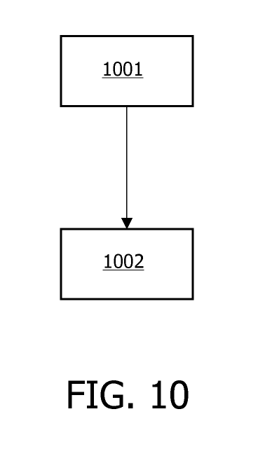

FIG. 10 schematically shows an implementation of the method according to the invention, wherein a monitored signal, indicative of movement of an external surface of the subject's thoracic wall, is sensed remotely,

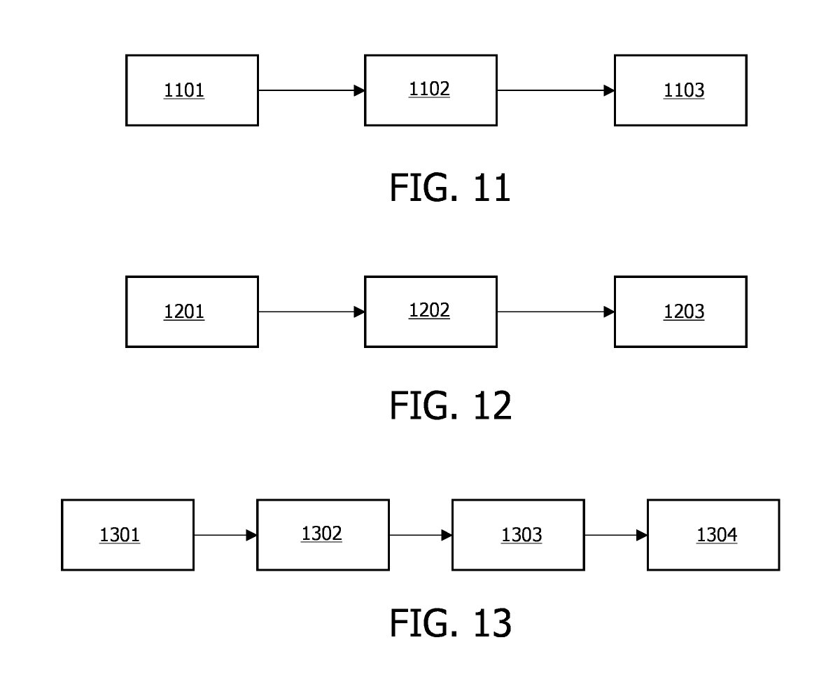

FIG. 11 schematically shows an implementation of the method according to the invention, wherein the monitored signal is processed to generate an output signal indicative of the heartbeat,

FIG. 12 schematically shows an implementation of the method according to the invention, wherein a measurement signal is transmitted towards the subject's thoracic wall, and wherein the monitored signal is generated from interactions of the measurement signal with the thoracic wall, and

FIG. 13 schematically shows an implementation of the method according to the invention, wherein the monitored signal is used to generate a triggering signal representative of a phase of the subject's heartbeat.

It may be noted that corresponding reference numerals used in the various Figures represent corresponding structures in the Figures.

FIG. 1 shows an embodiment of the invention where a thoracic wall 101 of a subject is monitored using a monitoring device 105 comprising a remote sensor 102. The input to the monitoring device 105 is a monitored signal 103 from the thoracic wall 101. The monitoring device 105 outputs an output signal 104.

The monitoring device 105 does not make direct physical contact with the patient, and the monitored signal 103 is received contactlessly. The monitoring device 105 could be, for example, a high-resolution, high-speed video camera that captures a movie of the chest wall 101. A video camera system with a frame capture rate of 100 frames per second or more allows for a reliable detection of the heartbeat-induced movements of the chest wall. As the effect of the heart motion on the chest wall is of the order of 500 microns, a video camera with a resolution of 25 microns would be sufficient to spatially resolve the movement on the chest wall resulting from the heartbeat.

FIG. 2 shows a further embodiment of the invention where the monitoring device 105 is used to study the heartbeat of a subject by studying its effect on the subject's chest wall 101. The monitoring device 105 comprises a remote sensor 204, which receives a monitored signal 103 without direct physical contact with the subject, and outputs a signal 203 to a processor 201 that processes the signal 203 to generate an output signal 202.

The remote sensor 204 is a high-resolution, high-speed video camera, the output of which 203 is sent to the processing unit 201. The processing unit 201 comprises a frame grabber, enabling the processor 201 to compare the incoming video pictures frame by frame, to detect movement of the thoracic wall. The processing unit 201 converts the signal 203 into a voltage or current signal, which is further fed to a display device, for example a screen or a monitor. The processor 201 alternatively comprises software that facilitates the identification of the object of interest, for example the patient's thorax, from the photographic images. Optical or other markers (not shown in the Figure) could be removably attached to the subject's thorax, for example by means of adhesive tape, wherein the markers could further facilitate the identification of the object of interest. The software performs a frame-to-frame comparison of the images to detect and monitor the heartbeat of the patient.

FIG. 3 shows an embodiment of the invention wherein the monitoring device 105 further comprises a transmitting device 304 that transmits incident radiation 301 towards the patient's chest wall 101. The monitoring device 105 also comprises a remote receiver or sensor 305 that receives a monitored signal 302 from the patient's chest wall 101. The sensing device 305 outputs an output signal 303.

The transmitting device 304 is a light source that illuminates the chest wall region of the patient. The remote sensing module 305 is a video camera, as explained in the description of FIG. 1. Alternatively, the sensing device 305 is a high-speed camera that captures snapshots of the thoracic wall. For example, a camera capable of taking 100 photographs per second could sufficiently resolve the effect of individual heartbeats on the thoracic wall. The temporal resolution (the frame rate) of the camera could be adjusted based on a priori knowledge, or an estimate, of the subject's average heart rate. For example, during the intervals between heartbeats the frame rate could be reduced. Shortly before the next heartbeat is predicted to start, the frame rate could be increased to high-speed in order to precisely capture the motion and localize it in the time domain.

FIG. 4 shows an embodiment of the invention wherein the monitoring device 105 comprises a remote receiver 404, connected to a triggering circuit 401 through a processing circuit 405. The remote sensor 404 receives a monitored signal 403 from the subject's thoracic wall 101, and the triggering circuit 401 outputs a triggering signal 402.

The remote sensor 404 is a high-speed photographic camera capable of capturing typically 100 frames or more per second. Each captured frame is compared with a consecutive frame to detect minute changes in the position of the chest wall, the comparison being done by the processing unit 405. Alternatively, the remote sensor 404 could be a video camera with a high spatial and temporal resolution, as explained in the description of FIG. 1. It may be sufficient to capture the photographic or video pictures in ambient light conditions. It may alternatively be advantageous to have a high-intensity light source that emits light towards the subject's thoracic wall 101. It may also be advantageous to use only a small section of the light spectrum by means of introducing an optical filter in front of the camera, e.g. an IR filter. The emitted light is the measurement signal, while the light reflected from the surface of the subject's thoracic wall 101 is the monitored signal.

The processing module could further comprise hardware or software to predict the next heartbeat, based on an average heart rate computed over a period of time. The heart rate varies slightly throughout the respiratory cycle, typically increasing slightly while inhaling and decreasing while exhaling. In addition to varying with the respiratory phase (called sinus arrhythmia), the heartbeat typically has an intrinsic variability. In either of the above cases, it is advantageous to use information from previous heartbeats in order to predict the next heartbeat more precisely.

The triggering circuit 401 generates a triggering signal 402 at a particular phase of the heartbeat. The phase of the heart could be calculated from time elapsed after a heartbeat is detected by the monitoring device 105. For example, at an average heart rate of 72 beats per minutes, one heart cycle lasts approximately 83 ms. The ventricular contraction phase occurs about 10 ms into the cardiac cycle, assuming the atrial contraction phase as the starting point of the cardiac cycle. The ventricular contraction phase will likely be the easiest to detect extracorporally, as the heart displacement is maximal during this phase, thus producing the maximum impact on the thoracic wall. Once the heartbeat is detected at the ventricular contraction phase, the other phases of the heart cycle can be worked out based on the time elapsed after the detected ventricular phase. For example, the ventricular diastolic phase of the heart occurs approximately 30 ms after the ventricular contraction phase. The triggering signal 402 is used to synchronize the acquisition of data in an imaging system, for example an MR system or a CT system or a 3D X-ray angiography system.

FIG. 5 shows an embodiment of the invention wherein the monitoring device 105 comprises a transmitter 502 that transmits either EM or ultrasonic radiation 501 towards the subject's thoracic wall 101. A remote receiver 507 receives EM or ultrasonic waves 503 reflected by the object. The processing circuit 508 comprises a mixer 504, which receives input from both the transmitter 502 and the receiver 507. The mixed signal is filtered using a low-pass filter 505 to generate an output signal 506.

The main lobe of the transmitted or measurement waves emitted is directed towards the object of interest, which in this case is the subject's thoracic wall. The frequency of those EM or ultrasonic waves that are reflected by the object is shifted with respect to the frequency of the transmitted waves. The frequency shift f_{Doppler} is related to the velocity of the object of interest according to the well-known equation

$${f}_{Doppler} = \pm {f}_{0} \cdot \frac{2 \cdot v}{c} ,$$

with f₀ being the frequency of the EM or ultrasonic wave emitted by the transmitter, c being the propagation velocity of the EM wave or the ultrasonic wave, respectively, and ν being the velocity of the object approaching the transmitter or departing from it, resulting in a positive or negative frequency shift, respectively. Mixing or multiplying the measurement signal 501 with the monitored signal 503 and low-pass filtering the mixed signal yields a signal 506 with the frequency f_{Doppler} at the output, without regard to whether the Doppler frequency shift of the received signal is positive or negative with respect to the frequency of the transmitted signal.

At f₀=1 GHz, for example, there will be a frequency shift of 0.67 Hz at the signal output if the object of interest moves with a constant velocity of 0.1 meter per second. As the motion of the skin surface induced by every heartbeat will last only for a fraction of a second, even if we assume that it is a motion with constant velocity during this short time, we will not see a full sine wave period of f_{Doppler} at the output. Rather, it is more reasonable to expect a single peak on the output signal at every heartbeat.

FIG. 6 shows a diagrammatic representation of the movement of the thoracic wall of a patient, wherein the effect of the patient's heartbeats 601 is shown superimposed on the chest wall motion caused by breathing 602.

The respiratory motion of the chest could produce an approximately sine-shaped signal 602 with a very low frequency, for example about 0.2 Hz, and superimposed on this signal produced by the respiratory motion would be the peaks 601 induced by the heartbeats. Thus, sudden changes in signal indicate heartbeat-induced motion, whereas slower changes can be attributed to respiration.

FIG. 7 shows an embodiment of the invention wherein the monitoring device 105 comprises a transmitter 702 to transmit visible EM radiation, an optical shearography system 707 connected to a processing unit 708 comprising a buffering medium 704 and a comparator device 705. The incident radiation or light 701 is reflected from the patient's thoracic wall 101, as the monitored signal 709. The optical shearography system outputs a time series of shearograms 703, which are processed by the processor 708. The processing unit 708 outputs the output signal 706.

In one embodiment, the shearographic sensor unit uses widened laser light for measuring the movements of the patient's thorax. Preferably, high-performance semiconductor laser devices are used. As a recording device, a CCD camera is used. By means of the optical system 707, a series of shearograms 703 is continually generated. The shearograms are stored in the buffering medium 704, and consecutive shearograms are compared to each other by the comparator 705. The processing unit produces difference images that contain the information about the movements of the patient's thorax. Alternative to a laser light source, light sources emitting EM radiation in the infrared, visible or ultraviolet wavelengths could be used to generate the shearograms.

FIG. 8 shows an embodiment of the invention wherein the monitoring device 105 comprises a sensor device 802 that senses signals 801 from the thorax 101 of a subject. The sensor device 802 is connected to a processor 803 comprising a frame grabber 807, a buffering medium 804, a motion analysis unit 805 and a heartbeat detection unit 809. The frame grabber 807 outputs a time series of stereographic images 808, and the processing unit 803 outputs an output signal 806 that is representative of the heartbeat of the subject.

If an object is photographed from two different positions, the line between the two projection centres is called the “base”. If both cameras have viewing directions that are parallel to each other and in a 90° angle to the base (the so-called “normal case”), then they have similar properties as the human eyes producing two images on two retinas.

Therefore, the overlapping area of these two images, which are called a “stereopair”, can be seen in three dimensions, simulating human stereoscopic vision. In practice, a stereopair can be produced with a single camera from two positions or by using a stereoscopic camera. However, compared to a single channel camera, using a stereoscopic camera system has the advantage of an enhanced assessment of the size, the distance and consequently also the movement of the monitored object. Typically, a stereoscopic camera consists of two cameras mounted at two ends of a bar, which has a precisely calibrated length (e.g. 40 cm). This bar functions as the base. Both cameras have the same geometric properties. As required for 3D vision, they have viewing directions that are parallel to each other and in a 90° angle to the base.

In an exemplary embodiment, a stereoscopic camera 802 monitors the patient's thorax 101. The output of the stereoscopic camera system is sent to a frame grabber circuit 807 that captures frames and generates a series of stereoscopic images 808 of the thorax 101. The images may be stored in a buffering medium 804 before being sent to a motion analysis unit 805 and a heartbeat detection unit 809. The motion analysis unit 805 and the heartbeat detection unit 809 could be implemented in either hardware or software or a combination of the two. Consecutive image captures taken during the measurement will show a change in image characteristics due to the movement of the heart. The motion analysis unit 805, in combination with the heartbeat detection unit 809, detects the change in image characteristics. The processing unit 803 thus generates an output signal 806 that is representative of the heartbeat of the subject.

FIG. 9 shows a system, for example, an MR system or a CT system or a cardiac 3D X-ray angiography system arranged to acquire image data from a patient. A remote sensor 404 receives a monitored signal 403 from a patient's thoracic wall 101. The monitored signal 403 is sent to a processing unit 405. The output of the processing unit 405 is sent to a triggering device 401 that generates a triggering signal 402. The imaging system 901 comprises a synchronization circuit 902 capable of utilizing the triggering signal 402 to initiate data acquisition on an imaging device 903. Additional information on using a triggering signal to synchronize data acquisition in an MR system may be obtained from Wendt R E, Rokey R, Vick G W, et al., “Electrocardiographic gating and monitoring in NMR imaging”, Magnetic Resonance Imaging, Vol. 6, Pg. 89-95 (1988), in a CT system from Schoepf U, Becker C R, Bruening RD et al., “Electrocardiographically Gated Thin-Section CT of the Lung”, Radiology, Vol. 212, Pp. 649-654 (1999), and in a cardiac 3D X-ray angiography system from Aschenbach S, Ulzheimer S, Baum U, et al., “Noninvasive Coronary Angiography by Retrospectively ECG-gated Multislice Spiral CT”, Circulation, Vol. 102, Pp. 2823-2828 (2000).

As may be noted in the above quoted references, data acquisition on the imaging system is synchronized to the heartbeat of a subject, the heartbeat being detected using an electrocardiogram (ECG) device. A normal ECG uses metal wires to conduct the ECG signal. These metal wires could introduce artifacts in MR images, thereby degrading image quality. It is thus advantageous to use a triggering signal derived from a remote sensing technique as in the invention, for triggering data acquisition on an MR system. In the case of CT or X-ray angiography, triggering data acquisition using a signal that is sensed in a non-contact manner, as outlined in the invention, provides the advantage of easier handling of the patient, as no ECG leads need to be applied to the patient.

FIG. 10 shows an implementation of the method according to the invention, the method comprising a step 1001 of receiving a monitored signal and a step 1002 of generating an output signal that is indicative of a heartbeat of a patient. The monitored signal is received without direct physical contact with the patient, in the sensing step 1001.

FIG. 11 shows a further implementation of the method according to the invention, the method comprising a step 1101 of receiving a monitored signal from the thoracic wall of a patient in a non-contact manner, a step 1102 of processing the monitored signal, and a step 1103 of generating an output signal that is indicative of the heartbeat of the patient.

FIG. 12 shows a further implementation of the method according to the invention, the method comprising a step 1201 of transmitting a measurement signal towards a patient, a step 1202 of receiving a monitored signal from the thoracic wall of the patient, and a step 1203 of generating an output signal that is indicative of a heartbeat of the patient, wherein the monitored signal is generated from interactions of the measurement signal with the patient's thoracic wall.

FIG. 13 shows a further implementation of the method according to the invention, the method comprising a step 1301 of receiving a monitored signal from the thoracic wall of a patient, a step 1302 of processing the monitored signal, a step 1303 of generating an output signal that is indicative of a heartbeat of a patient, and a step 1304 of generating a triggering signal that is indicative of a phase of the heartbeat of the patient.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The invention can be implemented by means of hardware comprising several distinct elements, and by means of a suitably programmed computer. In the system claims enumerating several means, several of these means can be embodied by one and the same item of computer-readable software or hardware. The mere fact that certain measures are recited in mutually different dependent claims does not indicate that a combination of these measures cannot be used to advantage.

1. A monitoring device (105) for remotely monitoring a heartbeat of a subject, the monitoring device comprising:
a receiver (105) arranged to receive a monitored signal (103) indicative of movement of an external surface of the subject's thoracic wall (101);
a processor (201) for processing the monitored signal (103) to generate an output signal (202) indicative of the heartbeat;
a triggering device (401) arranged to access the output signal (202) to generate a triggering signal (402) representative of a phase of the heartbeat; and
a utilization device arranged to utilize the triggering signal (402) to synchronize the acquisition of physiological data of the subject to the phase of the subject's heartbeat.

2. (canceled)

3. The monitoring device for remotely monitoring a heartbeat of a subject as claimed in claim 1, the monitoring device further comprising
a transmitter (304) for transmitting a measurement signal (301) towards the subject's thoracic wall (101), wherein
when in operation, the measurement signal (301) interacts with the thoracic wall (101) to generate the monitored signal (103).

4. (canceled)

5. The monitoring device for remotely monitoring a heartbeat of a subject as claimed in claim 3, wherein
the measurement signal is electromagnetic radiation,
the monitored signal is the reflected electromagnetic radiation, and
the output signal is the Doppler shift in frequencies between the measurement signal and the monitored signal.

6. The monitoring device for remotely monitoring a heartbeat of a subject as claimed in claim 3, wherein
the measurement signal is optical electromagnetic radiation,
the monitored signal is reflected optical electromagnetic radiation that is further processed to yield a time series of shearograms of the subject's thoracic wall and,
the output signal is obtained by comparing consecutive shearograms in the time series.

7. The monitoring device for remotely monitoring a heartbeat of a subject as claimed in claim 1, wherein
the monitored signal is optical electromagnetic radiation that is further processed to yield a time series of stereoscopic images, and
the output signal is obtained by comparing consecutive stereoscopic images in the time series.

8. The monitoring device for remotely monitoring a heartbeat of a subject as claimed in claim 3, wherein
the measurement signal is ultrasound radiation,
the monitored signal is the reflected ultrasound radiation, and
the output signal is the Doppler shift between the measurement signal and the monitored signal.

9. (canceled)

10. A method of remote monitoring of a heartbeat of a subject, comprising:
transmitting a measurement signal (301) towards the subject's thoracic wall (101) to produce a monitored signal from an interaction with the thoracic wall;
receiving the monitored signal indicative of a movement of an external surface of the subject's thoracic wall, the monitored signal being sensed remotely
processing the received signal to generate an output signal indicative of the heartbeat;
using the processed signal to generate a triggering signal representative of a phase of the subject's heartbeat; and
using the triggering signal to acquire physiological data of the subject in synchronism with a phase of the subject's heartbeat.

11.-15. (canceled)
